(19) United States
(12) Patent Application Publication
(10) Pub. No.: US 2013/0325006 A1
(43) Pub. Date: Dec. 5, 2013

(54) ARTICULATED INTRAMEDULLARY NAIL

(71) Applicant: Acumed LLC, Hillsboro, OR (US)

(72) Inventors: James W. Michelinie, Portland, OR (US); Mark Clark, Portland, OR (US); Matthew J. Ryan, Beaverton, OR (US)

(21) Appl. No.: 13/905,582

(22) Filed: May 30, 2013

Related U.S. Application Data
(60) Provisional application No. 61/653,316, filed on May 30, 2012.

(51) Int. Cl. A61B 17/72

(52) U.S. Cl. CPC A61B 17/7291
USPC 606/62

(57) ABSTRACT

System, including methods, apparatus, and kits, for bone fixation with an articulated nail. The nail may have a first segment and a second segment that extend from respective opposite ends of the nail to a joint region that connects the segments. Each segment may define one or more transverse apertures configured to receive a fastener that attaches the segment to bone. The joint region may have a movable configuration that permits pivotal rearrangement of the segments relative to each other and a locked configuration that fixes the segments relative to each other.

### CROSS-REFERENCE TO PRIORITY APPLICATION

This application is based upon and claims the benefit under 35 U.S.C. §119(e) of U.S. Provisional Patent Application Ser. No. 61/653,316, filed May 30, 2012, which is incorporated herein by reference in its entirety for all purposes.

### INTRODUCTION

The human skeleton is composed of 206 individual bones connected to one another by joints. Each joint determines the relative mobility of two or more adjacent bones. For example, synovial joints are found between bones having the greatest freedom of motion and enable bones of the appendages to move relative to one another and the axial skeleton.

Synovial joints can become damaged with age, overuse, or traumatic injury, among others, often resulting in chronic pain. A damaged joint may be treated surgically by partial or total arthroplasty (joint replacement) or by arthrodesis, which fuses bones at the joint.

Joint fusion may be performed by ablation of cartilage between bones, followed by bone fixation to promote bony union. Fixation can be provided by a fixation device spanning the joint, such as a plate, screws, a nail, or a combination thereof, among others.

Intramedullary nails for fusion are fixation devices configured to be received in bones to be fused. In a standard installation, the medullary cavity of each bone is accessed from an end or side of the bone, and then each cavity may be enlarged somewhat to permit the nail to fit into the cavity. After placement into the bones, the nail may be secured in position on opposing sides the joint. For example, the nail may include a plurality of transverse apertures that receive bone screws.

An intramedullary nail can be used to fix the tibiotalocalcaneal (TTC) junction between the bones of the leg and the foot. The TTC junction is a common site of joint wear and pain. A TTC fusion can be performed with a nail extending through three bones forming the TTC junction, namely, the tibia (the larger bone of the lower leg), the talus, and the calcaneus (the heel bone). These three bones meet at two interfaces of the TTC junction, namely, the ankle joint and the subtalar joint.

The ankle joint is a mortise joint created by articulation of the lower leg bones, the tibia and fibula, with the talus. The ankle joint permits flexion, i.e., dorsiflexion and plantar flexion, to respectively decrease and increase the angle formed between the leg and the foot, by pivotal motion generally in a sagittal plane (about one or more axes oriented generally medial-laterally).

The subtalar joint is created by articulation of the talus with the calcaneus. The subtalar joint permits valgus and varus motion of the calcaneus, to produce eversion and inversion of the foot, respectively, by pivotal motion generally in a coronal plane (about one or more axes generally parallel to the long axis of the foot).

Tibiotalocalcaneal fusion with an intramedullary nail is indicated for patients with ankle and subtalar arthrosis, post-traumatic arthritis of the ankle, talar avascular necrosis, failed total ankle arthroplasty, Charcot foot, complex hindfoot deformity, or severe fracture at the ankle, among others. To achieve fixation, an intramedullary nail can be inserted in a retrograde direction, from a plantar surface of the foot, through the calcaneus and the talus, and into the tibia. Screws then can be placed to extend into the nail from each bone.

An improved intramedullary nail is needed for performing TTC fusion and/or fixation of other bones.

### SUMMARY

The present disclosure provides a system, including methods, apparatus, and kits, for bone fixation with an articulated nail. The nail may have a first segment and a second segment that extend from respective opposite ends of the nail to a joint region that connects the segments. Each segment may define one or more transverse apertures configured to receive a fastener that attaches the segment to bone. The joint region may have a movable configuration that permits pivotal rearrangement of the segments relative to each other and a locked configuration that fixes the segments relative to each other.

### BRIEF DESCRIPTION OF THE DRAWINGS

FIG. 1 is a medial view of the bones of the lower left leg and left foot with an exemplary articulated nail installed in the calcaneal bone, the talar bone, and the tibial bone to promote a tibiotalocalcaneal fusion, in accordance with aspects of the present disclosure.

FIG. 2 is a pair of side views of the articulated nail of FIG. 1 with a joint region of the nail disposed either in a movable configuration (on the left) or a fixed (i.e., locked) configuration (on the right), in accordance with aspects of the present disclosure.

FIG. 3 is a side view of the articulated nail of FIG. 1 with the nail in a linear configuration.

FIG. 4 is a sectional view of the articulated nail of FIG. 3, taken generally along line 4-4 of FIG. 3.

FIG. 5 is a fragmentary sectional view of the articulated nail of FIG. 3, taken generally as in FIG. 4 with a tool received in the nail and engaged with a locking member of the nail to illustrate adjustment between the movable and fixed configurations of the joint region of the nail.

FIG. 6 is a fragmentary sectional view of the articulated nail of FIG. 3, taken generally around the joint region of the nail with the joint region in a movable configuration.

FIG. 7 is another fragmentary sectional view of the articulated nail of FIG. 3, taken generally as in FIG. 6 but viewed with a small angular offset toward the trailing end of the nail, with the locking member not sectioned, and with the joint region in a fixed configuration.

FIG. 8 is yet another fragmentary sectional view of the articulated nail of FIG. 3, taken generally as in FIG. 6 but with the nail in an angular configuration and with the joint region locked to fix the nail in the angular configuration.

FIGS. 9-14 are fragmentary medial views of the bones of FIG. 1 taken during performance of an exemplary method of installing the articulated nail of FIG. 1 in an arthrodesis procedure for fusion of the tibial, talar, and calcaneal bones, in accordance with aspects of the present disclosure.

FIGS. 15-18 are fragmentary anterior views of a fractured distal femur taken during performance of an exemplary method of installing an articulated intramedullary nail to fix a single bone, in accordance with aspects of the present disclosure.

FIGS. 19-22 are fragmentary anterior views of a fibula and a proximally fractured tibia taken during performance of an exemplary method of installing an articulated intramedullary nail in the tibia to fix the bone, in accordance with aspects of the present disclosure.

FIGS. 23-25 are fragmentary sectional views of another exemplary articulated nail, taken generally around the adjustable joint of the nail, in accordance with aspects of the present disclosure.

FIG. 26 is a fragmentary sectional view of yet another exemplary articulated nail, taken generally around the adjustable joint of the nail, in accordance with aspects of the present disclosure.

FIG. 27 is another fragmentary sectional view of the articulated nail of FIG. 26, taken generally as in FIG. 26 but with the nail sectioned in a quadrant and with the locking member incompletely sectioned.

FIG. 28 is a view of the locking member of FIG. 27 taken in isolation.

FIG. 29 is a fragmentary sectional view of still another exemplary articulated nail, taken generally around the adjustable joint of the nail, in accordance with aspects of the present disclosure.

FIG. 30 is a fragmentary view of one of the segments of the nail of FIG. 29, taken toward a joint surface of the segment and illustrating exemplary projections extending from the joint surface.

FIG. 30A is a fragmentary view of another embodiment of the nail segment depicted in FIG. 30, with the nail segment having ribs formed on a joint surface to resist slippage, in accordance with aspects of the present disclosure.

FIG. 31 is a fragmentary sectional view of an exemplary articulated nail having a cam mechanism for adjustment of the joint of the nail between movable and fixed configurations, with the view taken generally around the joint of the nail and with the joint in a movable configuration, in accordance with aspects of the present disclosure.

FIG. 32 is another fragmentary sectional view of the nail of FIG. 31, taken with the joint in a fixed configuration.

FIG. 33 is a view of an actuator of the cam mechanism of FIG. 31.

FIG. 34 is a fragmentary sectional view of another exemplary articulated nail having a cam mechanism for adjustment of a joint of the nail between movable and fixed configurations, in accordance with aspects of the present disclosure.

FIG. 35 is a view of an actuator of the cam mechanism of FIG. 34.

FIG. 36 is a fragmentary side view of an exemplary articulated nail having a joint region composed of a pair of pivotable joints, in accordance with aspects of the present disclosure.

FIG. 37 is a fragmentary sectional view of the nail of FIG. 36.

FIG. 38 is another fragmentary side view of the articulated nail of FIG. 36.

FIG. 39 is a view of an exemplary biasing member (here, a leaf spring) that may be disposed at a joint of the nail of FIG. 36 to urge joint surface regions apart from one another.

FIG. 40 is a fragmentary, longitudinal sectional view of another exemplary articulated nail having a joint region composed of a pair of pivotable joints, with the joint region in a locked configuration, in accordance with aspects of the present disclosure.

FIG. 41 is a magnified portion of the sectional view of FIG. 40, taken generally around the region indicated at “41” in FIG. 40 to show sets of teeth that are meshed with each other one of the joints.

FIG. 42 is a fragmentary side view of selected components of an exemplary articulated nail having a joint region composed of three pivotable joints, in accordance with aspects of the present disclosure.

FIG. 43 is a side view of the articulated nail of FIG. 1 attached to an exemplary guide device, with the nail locked in a linear configuration, in accordance with aspects of the present disclosure.

FIG. 44 is a side view of the nail and guide device of FIG. 43, with the nail arranged in an angular configuration, in accordance with aspects of the present disclosure.

### DETAILED DESCRIPTION

The present disclosure provides a system, including methods, apparatus, and kits, for bone fixation with an articulated nail. The nail may have a first segment and a second segment that extend from respective opposite ends of the nail to a joint region that connects the segments. Each segment may define one or more transverse apertures configured to receive a fastener that attaches the segment to bone. The joint region may have a movable configuration that permits pivotal rearrangement of the segments relative to each other and a locked configuration that fixes the segments relative to each other.

A method of bone fixation is provided. The method may be performed with a nail having a first segment and a second segment that extend from respective opposite ends of the nail to a joint region that connects the segments. The nail may be inserted into bone with the nail locked in a linear configuration. Each of the segments may be attached to bone with one or more fasteners. The nail may be arranged, after insertion into the bone, in an angular configuration by moving at least one of the segments relative to the other segment at the joint region. The nail may be locked in the angular configuration.

The nail disclosed herein may have substantial advantages for TTC fusion. The primary goal of TTC fusion is to achieve a stable, pain-free foot in an optimal position through fusion of the ankle and subtalar joints. The desired positioning generally entails neutral flexion, 5 degrees of valgus rotation, and 5 to 10 degrees of external rotation. In some embodiments, the articulated nail disclosed herein can help achieve this desired positioning in a greater percentage of patients.

Further aspects of the present disclosure are presented in the following sections: (I) overview of an exemplary articulated nail system, (II) installation of an articulated nail for bone fusion, (III) installation of an articulated nail in a single bone, (IV) kits, and (V) examples.

### I. OVERVIEW OF AN EXEMPLARY ARTICULATED NAIL SYSTEM

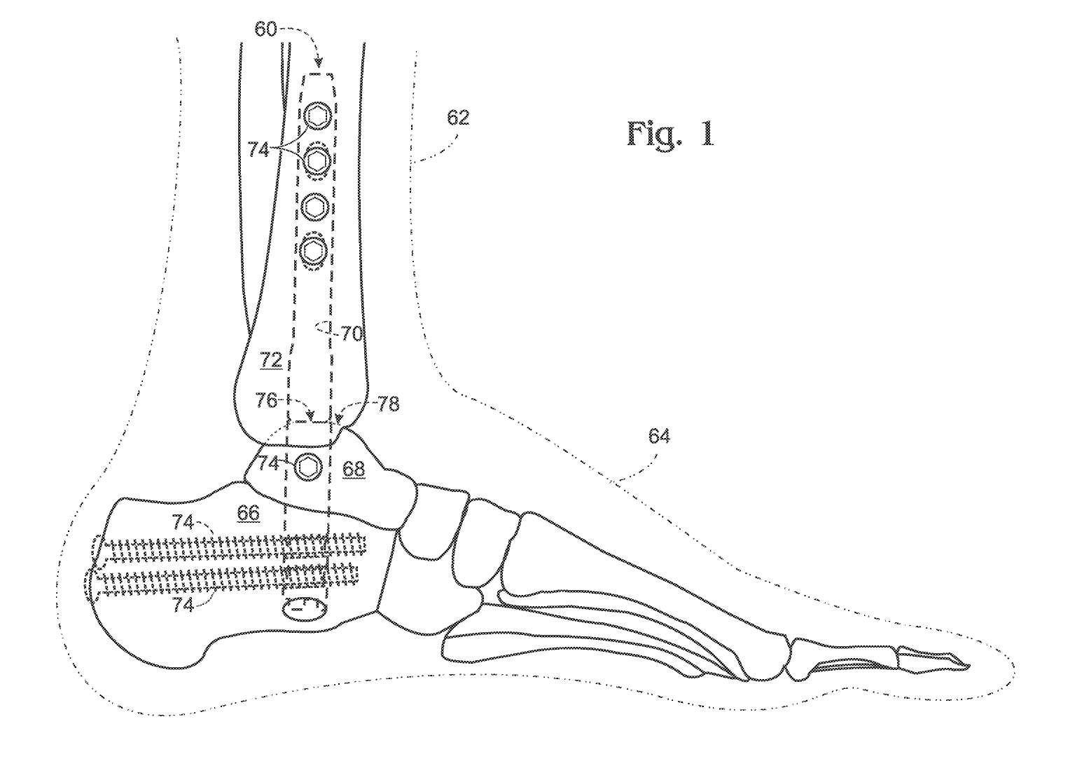

This section provides an overview of an exemplary bone fixation system that includes an articulated intramedullary nail 60; see FIGS. 1-8.

FIG. 1 shows the bones of a lower left leg 62 and a left foot 64 after installation of articulated nail 60. Here, the nail extends from a calcaneal bone 66 (the calcaneus or heel bone), through a talar bone 68 (the talus), and into a medullary cavity 70 of a tibial bone 72 (the tibia). In some cases, talar bone 68, if damaged substantially, may be removed before insertion of the nail. Nail 60 may be attached to each of bones 66, 68, and 72 with fasteners 74, such as bone screws, to hold the bones adjacent one another to promote a tibiotalocalcaneal fusion (i.e., a fusion of bones 66, 68, and 72).

Nail 60 includes a pivot region having a pivotable joint 76 (interchangeably termed an adjustable joint) that permits adjustment of the position of foot 64 relative to the leg 62 after nail insertion. Nail joint 76 may be positioned generally at an ankle joint 78, namely, generally between tibial bone 72 and talar bone 68. Joint 76 has a movable configuration that permits pivotal movement of segments of the nail relative to one another (e.g., with at least two or with three degrees of pivotal freedom) and a fixed (locked) configuration that prevents pivotal movement and fixes the geometry of the nail (e.g., in a linear configuration or an angular configuration).

The pivot region of the nail may permit the segments to be pivoted relative to each other in each of a pair of planes that are transverse (e.g., orthogonal) to each other and parallel to the long axis of the nail and/or a nail segment. The pivot region also may permit the segments to be pivoted relative to each other about a long axis of the nail and/or a nail segment. The pivot region may have only a single joint, as shown for nail 60, or two or more joints (e.g., see Example 5). Each joint may permit pivotal rearrangement of segments 92 and 94 relative to each other in only a single plane (e.g., permitting pivotal repositioning of a segment about only a single axis). Alternatively, each joint may permit the segments to be pivotally rearranged relative to each other in each of a pair of planes that are transverse (e.g., orthogonal) to each other. Stated another way, the joint region may permit pivotal rearrangement of the segments relative to each other about each of a pair of axes that are transverse (e.g., orthogonal) to each other. In some cases, the joint region may permit pivotal rearrangement about three axes that are orthogonal to each other.

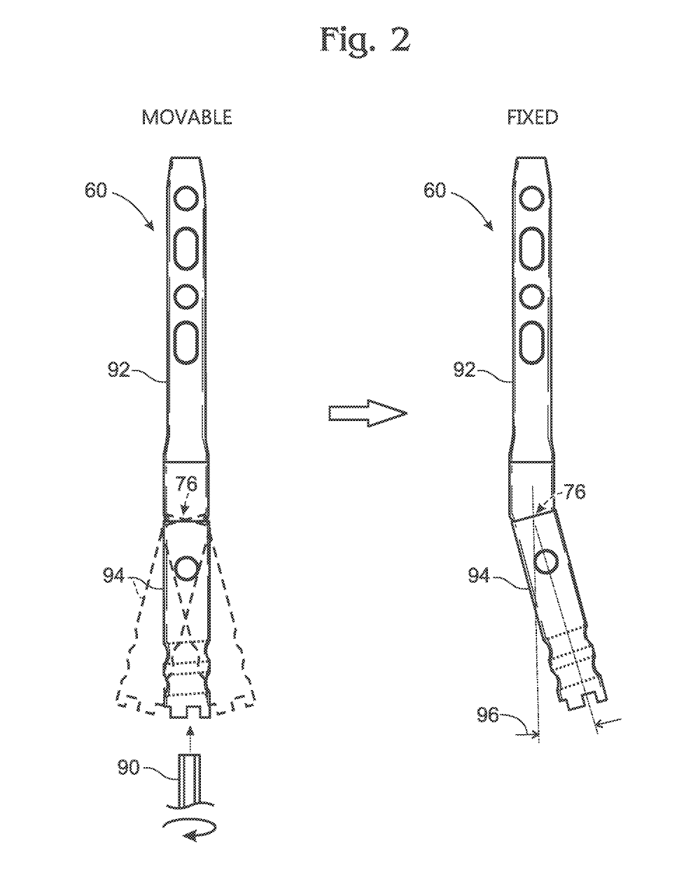

FIG. 2 shows a pair of side views of nail 60 with joint 76 disposed in a movable configuration (on the left) and a fixed configuration (on the right). The nail may be adjusted between the configurations with a tool 90 received in the nail from an end and/or a side of the nail.

Nail 60 may include a pair of elongate segments 92, 94 connected to one another by joint 76. The segments may extend from respective opposite ends of the nail and collectively may extend along at least a majority of the length of the nail (i.e., greater than 50% of the length), such as more than 70%, 80%, or 90% of the nail's length, among others. For example, here, segment 92 is a leading segment that extends to a leading boundary of the nail, segment 94 is a trailing segment that extends to a trailing boundary of the nail, and the segments collectively extend along the entire length of the nail. Each segment may be described as inflexible, meaning that the segment is substantially less flexible than the nail at the joint with the nail in a movable configuration. The segment may be described as nonarticulated, meaning that the segment lacks a mechanical joint that permits the shape of the segment to be altered.

Adjustable joint 76 may permit any range of pivotal motion of one segment relative to another segment of the nail. For example, joint 76 may permit the nail to flex through an angle 96 of at least about 2, 4, 6, 7.5, or 10 degrees, among others, relative to a coaxial arrangement of the segments. Stated differently, the nail may be bent selectively at the adjustable joint through a continuous range of angles (or only to discrete angles) at least up to 2, 4, 6, 7.5, or 10 (or more) degrees. One segment also may be turned about the long axis of the nail relative to the other segment. In sum, adjustable joint 76 may permit three degrees of pivotal freedom in a movable configuration and no degrees of pivotal freedom in a fixed configuration. In other embodiments, the nail may have a joint region composed of a plurality of joints, with each joint providing a different degree of pivotal freedom (see Example 5).

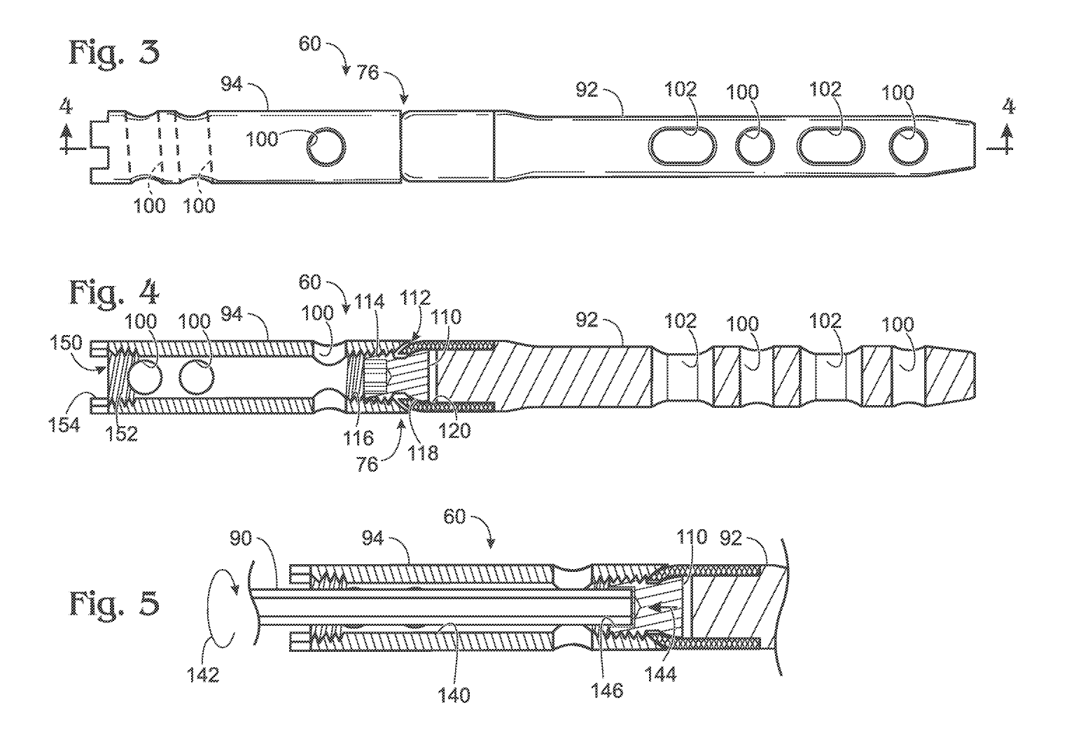

FIG. 3 shows a side view of nail 60 in a linear configuration. Each segment 92, 94 may define one or more transverse apertures (e.g., blind holes or through-holes) to receive fasteners that attach the segment to bone. Each transverse aperture defines an aperture axis that is transverse to a long axis of the nail and/or a nail segment that defines the aperture. Leading segment 92 and trailing segment 94 each may define one or more cylindrical or circular transverse apertures 100 and one or more elongated transverse apertures or slots 102. Each elongated transverse aperture may be elongated in a direction transverse (e.g., orthogonal) to a through-axis defined by the aperture. The transverse apertures within each segment may define parallel axes, as shown here for two apertures in the trailing segment and all apertures in the leading segment, or may define two or more axes that are not parallel to one another.

The segments of the nail may have any suitable size and shape. Each segment may or may not have a circular cross-section and thus may be at least generally cylindrical. The diameter of each segment may be at least generally uniform (e.g., as shown for trailing segment 94) or may vary along the length of the segment. For example, in the depicted embodiment, leading segment 92 tapers toward the leading boundary of the nail at two positions: a first position between the adjustable joint and the apertures and a second position adjacent the leading boundary. Each tapered region may or may not be substantially conical. The leading and trailing segments may have any suitable relative lengths according to the intended use of the nail. For example, the trailing segment may be shorter than the leading segment (as shown here), may be about the same length as the leading segment, or may be longer than the leading segment. Each segment may be linear or may have a predefined bend, such as a bend created during manufacture of the nail.

FIG. 4-8 show various longitudinal sectional views of nail 60. The nail may include a locking member 110 that is manipulable (e.g., rotatable) to adjust the adjustable joint between movable and fixed configurations. The locking member may extend from one segment to the other segment and may span a junction 112 formed between the segments (see FIG. 4). The locking member may have a threaded region 114 with an external thread that engages a complementary internal thread 116 formed in trailing segment 94 (see FIGS. 4 and 7). A head 118 may be formed on the locking member adjacent threaded region 114. The head may be captured in a cavity 120 defined by the trailing portion of leading segment 92, to connect the segments to each other in movable and fixed configurations of the adjustable joint.

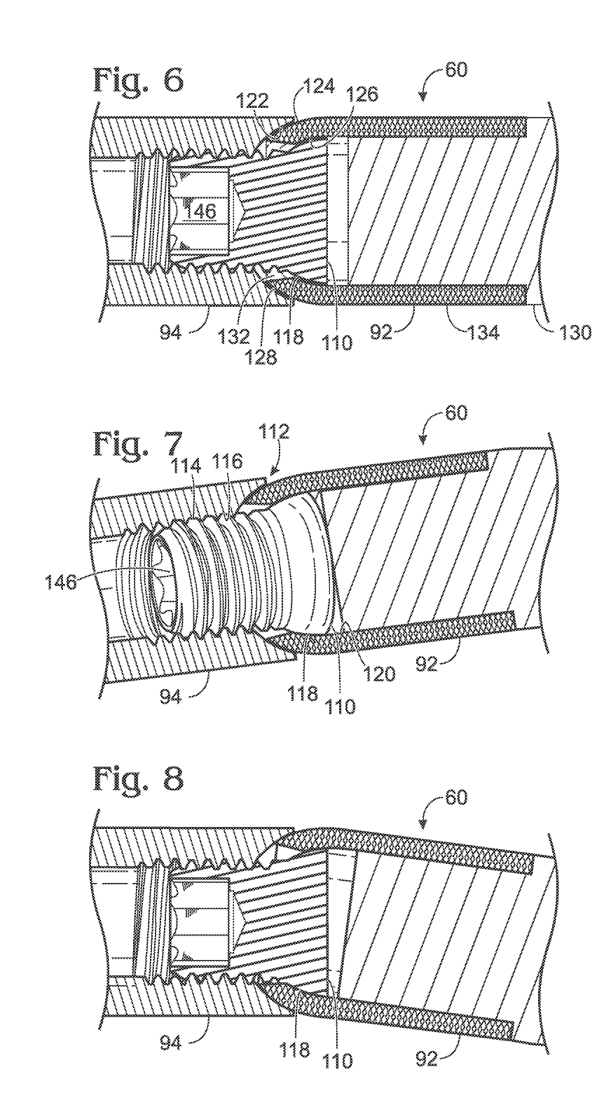

The joint region of the nail may include a first pair of complementary spherical surface regions 122, 124 (see FIG. 6). The surface regions may be provided by adjacent ends of the segments. The joint also may include a second pair of complementary spherical surface regions provided by head 118 and a wall 126 of cavity 120. The complementary surface regions of each pair permit pivotal motion about a pivot point with three degrees of freedom in the movable configuration of the adjustable joint (e.g., in FIGS. 4 and 6) and engage one another in the fixed configuration (e.g., in FIGS. 5, 7, and 8). The spherical surface regions may represent any suitable portion of a sphere, such as a frustrospherical portion, among others.

Spherical surface region 124 and wall 126 may be formed by a spherical flange 128 projecting from a body 130 of leading segment 92 near the trailing boundary thereof (see FIG. 6). The flange may be received in a gap 132 formed between spherical surface region 122 of trailing segment 94 and head 118 of locking member 110. Movement of the flange within the gap is permitted, to enable pivotal motion of the segments, when the locking member is disposed in a longitudinally advanced position, as in FIG. 6. However, movement of the flange within the gap is blocked, to fix the segments relative to one another, when the locking member is disposed in a retracted position that narrows the gap and compresses the flange between trailing segment 94 and head 118, as in FIGS. 7 and 8.

Flange 128 and body 130 of leading segment 92 may be provided by discrete pieces that are attached to one another, as shown for nail 60 in FIG. 6. For example, flange 128 may be formed by a sleeve 134 secured to body 130 by any suitable mechanism, such as a press fit, threaded engagement, fasteners (e.g., pins, rivets, screws, etc.), bonding, welding, or the like. Alternatively, the flange and body may be formed continuously with one another, as a monolithic component.

FIG. 5 illustrates exemplary adjustment of nail 60 to a fixed configuration. The tip of tool 90, interchangeably termed a driver, has been placed into operative engagement with locking member 110 from a trailing end of trailing segment 94 via an axial passage 140. The passage may extend through the segment and may be occupied at one end by the locking member. In the depicted configuration, tool 90 been used to turn the locking member, indicated by a rotation arrow at 142, to retract the locking member, indicated by a motion arrow at 144. The tool may be operatively engaged with a driver/tool engagement structure 146 (e.g., a recess, such as a polygonal recess, a slot, or a cross, among others) formed at one end of locking member 110 (also see FIGS. 6 and 7). Depending on the rotational direction in which the tool is turned, the locking member can be advanced or retracted, to respectively unlock or lock the adjustable joint of the nail.

Each joint of the nail may restrict relative motion of the nail segments in a locked configuration by any suitable mechanism. For example, as in nail 60, the joint may rely, at least predominantly, on frictional engagement between joint surface regions to lock the joint. In other cases, the joint may rely, at least predominantly, on mechanical engagement between joint surface regions to lock the joint (e.g., see Example 5). Mechanical engagement, as used herein, may be any contact that places a portion of one of the joint surface regions in the pivotal travel path of a portion of another joint surface region, to obstruct pivotal motion along the travel path. For example, one or two or more projections defined by one of the joint surface regions may be received in one or two or more recesses defined by the other joint surface region to block pivotal movement of the surface regions relative to each other.

FIG. 4 depicts attachment structure 150 formed near the trailing boundary of the nail. The attachment structure may include a threaded region 152 and a keyed region 154 to respectively provide threaded engagement with an instrument and restrict the rotational orientation of the instrument to a predetermined direction. Structure 150 may be utilized to attach any suitable instrument, such as a positioning jig and/or a guide device, to the nail. The instrument may, for example, be used to target a drill and/or a fastener to apertures of the nail and/or to externally mark the position of the adjustable joint with respect to bone (e.g., to facilitate placement of the adjustable joint near an anatomical joint and/or bone discontinuity (e.g., a fracture)). The nail may be plugged with a cap that engages threaded region 152 after the instrument has been disconnected.

The nail may have any suitable composition and other features. The nail may be formed of a biocompatible material, such as metal (e.g., stainless steel, titanium, cobalt-chrome, or the like), or a polymer (e.g., polyethylene, polyether ether ketone, or the like), among others. The nail may or may not be cannulated (i.e., provided with an axial bore that extends longitudinally through both segments and the locking member).

### II. INSTALLATION OF AN ARTICULATED NAIL FOR BONE FUSION

This section describes exemplary methods of fixing two or more bones with an articulated nail, such as in an arthrodesis procedure to fuse the bones; see FIGS. 9-14. The steps described in this section may be performed in any suitable combination and order, and may be combined with any other procedures described elsewhere herein.

An articulated nail may be selected for installation. The nail may have any suitable combination of the features described herein, such as a pair of segments connected by an adjustable joint region having one or more movable joints. Nail 60 of Section I (FIGS. 1-8) is selected here for illustration. However, any other articulated nail disclosed herein may be used instead.

At least two bones may be selected for fixation. The bones generally articulate with one another via an anatomical joint. Exemplary bones that may be selected include adjacent bones in the foot and/or lower leg, in the hand and/or wrist, or the like. Cartilage between the selected bones may be removed to facilitate fusion of the bones via bone growth.

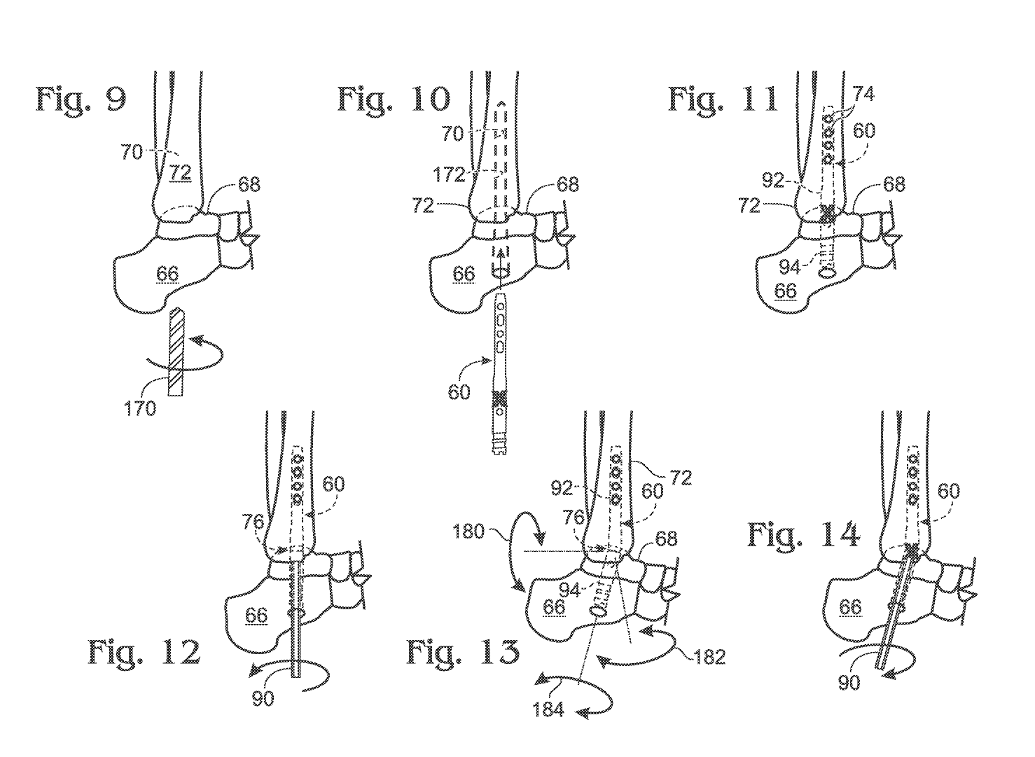

FIG. 9 shows calcaneus 66, talus 68, and tibia 72 selected for fixation. A hole-forming tool, such as a drill 170, may create a hole from the plantar surface of calcaneus 66, through the calcaneus and talus 68, and into medullary cavity 70 of tibia 72. The hole may be modified with additional instruments such as a broach, a reamer, or the like, to prepare a cavity 172 that is sized and shaped to receive nail 60 (see FIG. 10).

FIGS. 10 and 11 depict insertion of nail 60 into prepared cavity 172 of calcaneus 66, talus 68, and tibia 72. The nail may be placed through the calcaneus and talus and into medullary cavity 70 of tibia 72. Leading segment 92 then may be attached to tibia 72 with one or more fasteners 74 placed to extend into and/or through transverse apertures of the leading segment. Trailing segment 94 of the nail may not be attached to calcaneus 66 or talus 68 with fasteners at this point in the procedure, since the fasteners could obstruct access to the locking member of the adjustable joint, for adjustment between fixed and movable configurations. However, in some embodiments (e.g., see Example 4), the locking member can be accessed from the side of the nail, which permits placement of fasteners into the trailing segment of the nail for attachment to the calcaneus at this point in the procedure.

During nail insertion, adjustable joint of nail 60 may be in the fixed configuration, as indicated by an “X” over the joint. In exemplary embodiments, prepared cavity 172 is linear and the nail is inserted into the cavity with the nail locked in a linear configuration. An adjustable joint (and/or joint region) of the nail may be positioned generally between bones, such as positioned generally at the ankle joint formed between talus 68 and tibia 72. Placement of the adjustable joint between bones may allow the nail to be bent more easily to an angular configuration after the adjustable joint is unlocked.

Placement of fasteners into leading segment 92 and/or trailing segment 94 may be facilitated by a guide device attached to the nail, such as mounted at the trailing end of the trailing segment. The guide device may be capable of guiding fasteners 74 to apertures in the leading segment of the nail, particularly when the nail is in a straight, locked configuration, and before the nail is bent and/or twisted at the adjustable joint (if needed). Accordingly, insertion of the nail and attachment to one or more bones may be performed with the nail in a straight, locked configuration, before any change in the relative orientation of the leading and trailing segments (by bending the nail and/or twisting one segment relative to the other about the long axis), to allow proper targeting of apertures of the leading segment via the guide device attached to the trailing segment. Example 6 describes further aspects of guide devices that may be suitable.

FIG. 12 shows adjustable joint 76 of nail 60 being adjusted with driver 90 to place the joint in a movable configuration (also see FIG. 5). Rotation of driver 90 advances the locking member of the nail to render the nail bendable at the adjustable joint. Changing the adjustable joint to the movable configuration is optional during a surgical procedure. For example, if the surgeon is satisfied with the position of the foot relative to the leg after nail insertion, there is no need to unlock the adjustable joint. Instead, the surgeon can proceed with attachment of the trailing segment of the nail to the talus and calcaneus.

FIG. 13 illustrates adjustment of the orientation of trailing segment 94 relative to leading segment 92, and associated adjustment of the relative positions of bones 66, 68, and 72. Adjustable joint 76 in the movable configuration may permit trailing segment 94 to be pivoted with respect to leading segment 92 with one or more, at least two, or with three degrees of pivotal freedom, indicated schematically at 180, 182, and 184. Accordingly, the nail can be manipulated as needed to achieve a desired orientation of the foot relative to the leg. For example, pivotal motion indicated at 180 can adjust the varus/valgus position of the foot, at 182 can adjust the flexion of the foot, and at 184 can adjust the internal/external rotation of the foot.

FIG. 14 shows nail 60 being locked in an angular configuration by tool 90. The “X” over the adjustable joint of the nail indicates that the joint has been locked again.

### III. INSTALLATION OF AN ARTICULATED NAIL IN A SINGLE BONE

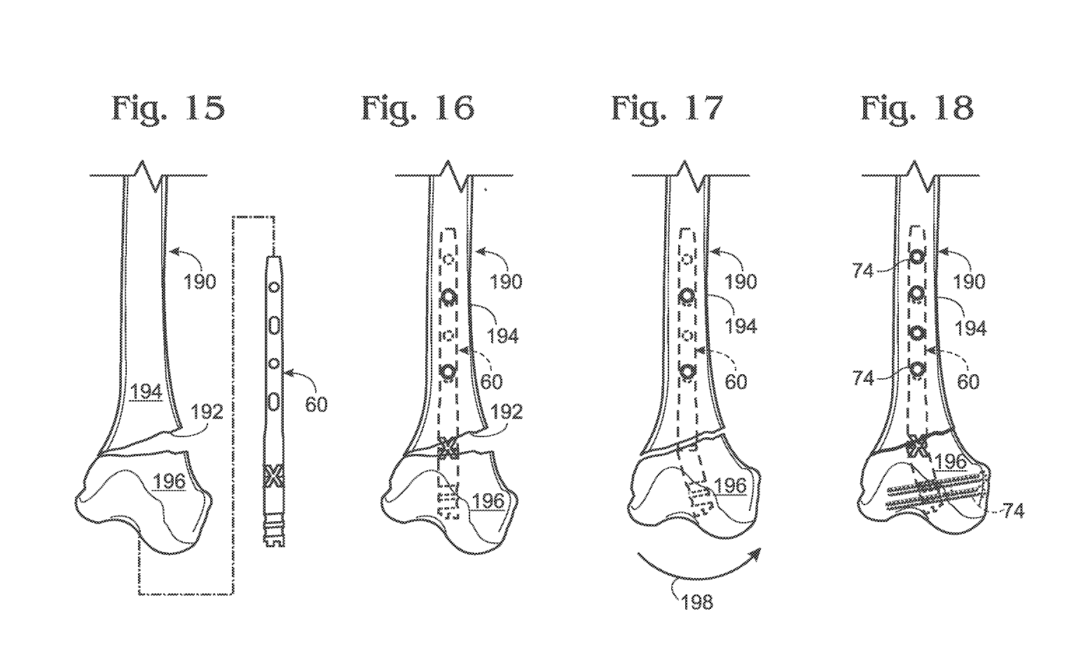

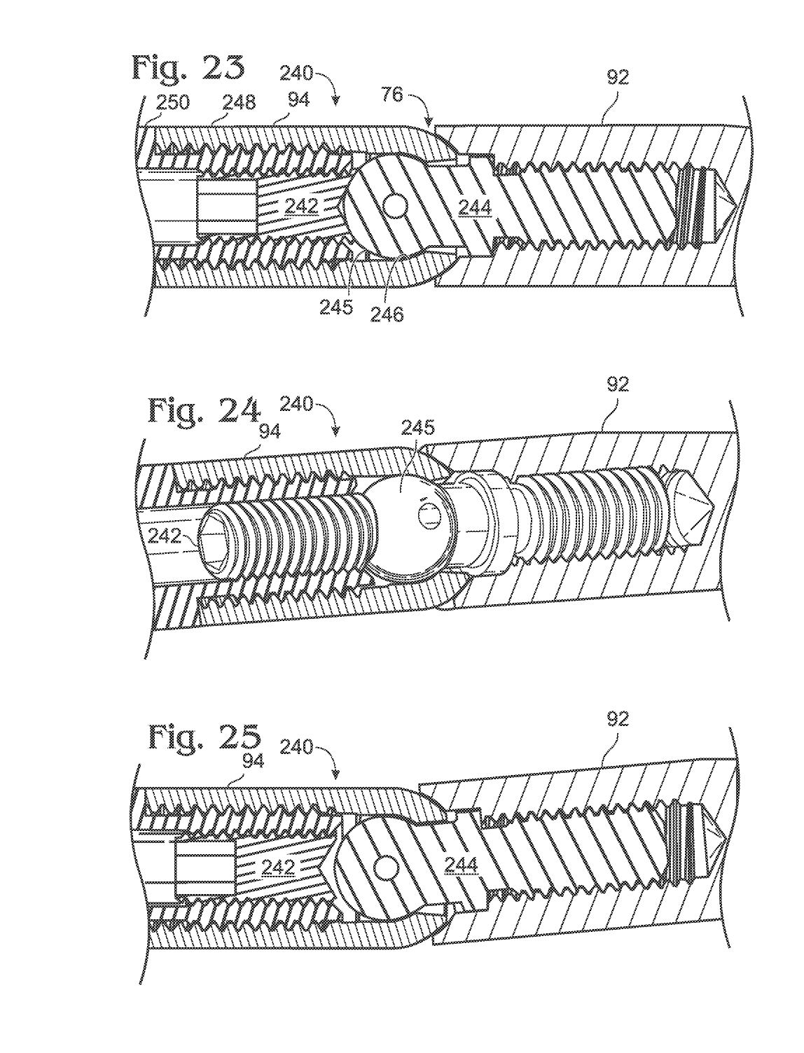

This section describes exemplary methods of fixing a single bone, such as a broken or cut bone, with an articulated nail; see FIGS. 15-22. The steps described in this section may be performed in any suitable combination and order, with any suitable articulated nail, and may be combined with any other procedures described elsewhere herein.

FIGS. 15-18 show fragmentary anterior views of a fractured distal femur 190 taken during performance of an exemplary method of installing an articulated intramedullary nail. Femur may have a discontinuity, such as a fracture 192 near the knee, to produce a proximal fragment 194 and a distal fragment 196. The goal is to reduce the fractured femur and fix fragments 194, 196 in proper alignment with one another, to restore knee function.

FIG. 15 shows nail 60 before insertion. The medullary cavity of the femur may be accessed, such as with a drill or punch, from the distal end of the femur, and then may be prepared, such as with a broach or reamer, to receive the nail.

FIG. 16 shows nail 60 after insertion into the medullary cavity of the femur. The nail may be inserted with the adjustable joint of the nail in a fixed (or movable) configuration. Here, the adjustable joint is in a fixed linear configuration, as indicated by the “X” over the adjustable joint. Insertion may dispose the nail within femur 190, with the nail spanning fracture 192 and with the adjustable joint of the nail positioned generally between fragments 194, 196.

FIG. 17 shows nail 60 in an angular configuration produced by unlocking the adjustable joint and pivoting the trailing segment with respect to the leading segment of the nail, indicated by a motion arrow at 198. The nail may be flexed by repositioning distal fragment 196 with respect to proximal fragment 194 of the femur. As shown here, this repositioning may improve the reduction of the fractured bone and may close the gap between bone fragments. The gap may be closed further by compressing the bone longitudinally at any suitable time during the procedure.

FIG. 18 shows nail 60 fully installed and re-locked, but now in a flexed configuration. Fasteners 74 may be placed into distal fragment 196 to attach the distal fragment to the trailing segment of the nail. Also, one or more additional fasteners 74 may be placed into proximal fragment 194 of the femur to further restrict longitudinal motion of the leading segment of the nail with respect to the proximal fragment of the femur. However, in some embodiments, the apertures of the leading segment may be difficult to target accurately after the nail has been placed in an angular configuration.

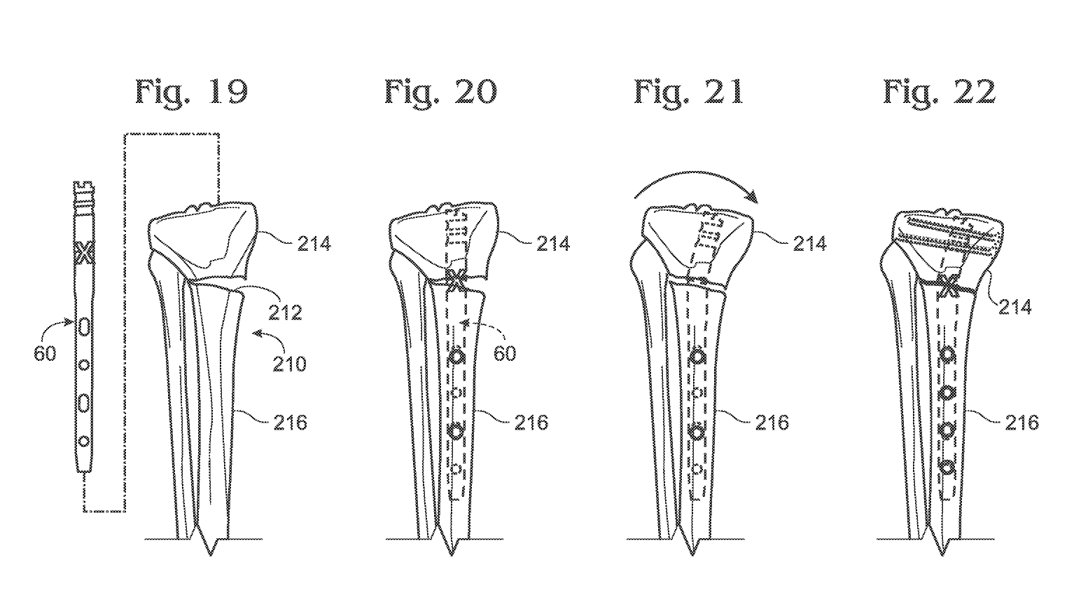

FIGS. 19-22 show fragmentary anterior views of a fractured proximal tibia 210 taken during performance of an exemplary method of installing an articulated intramedullary nail. Tibia 210 may have a discontinuity, such as a fracture 212 near the knee, to create a proximal fragment 214 and a distal fragment 216. The goal is to reduce the fractured tibia and fix fragments 214, 216 in alignment with each other, to restore knee function. Nail 60 may be installed in the tibia generally as described above for installation into the femur.

Any suitable fractured/cut bone may be fixed with an articulated intramedullary nail, as disclosed herein. The bone may be a long bone, such as a humerus, ulna, radius, fibula, or the like, and may have a discontinuity near either end of the bone or centrally along the bone. Other exemplary bones that may be fixed with an articulated intramedullary nail include a clavicle, a rib, a mandible, a carpal bone, a metacarpal bone, a tarsal bone, a metatarsal bone, a phalange, or the like.

### IV. KITS

The articulated nail may be provided in a kit. The kit may include any suitable combination of the following: one or more articulated nails (e.g., nails of different length, diameters, etc.), fasteners to secure the nail to bone, a tool to adjust the nail between movable and fixed configurations, a guide/jig that attaches to the nail to define paths (guide axes) for drilling and fastener placement, one or more instruments to prepare bone for receiving the nail (e.g., a punch, drill, broach, reamer, etc.), and instructions for use, among others.

### V. EXAMPLES

This section presents selected aspects and embodiments of the present disclosure related to exemplary articulated nails having an adjustable (movable) configuration and a fixed (locked) configuration. These aspects and embodiments are intended for illustration and should not limit the entire scope of the present disclosure. Any elements, aspects, or features of the nail embodiments disclosed in this section may be combined with each other and/or with any combination of elements, aspects, or features of the nails described elsewhere in the present disclosure (e.g., in Sections I through IV).

### Example 1

### Articulated Nail with Inverted Arrangement of Joint Surfaces

This example describes an exemplary articulated nail 240 with a generally inverted arrangement of joint surfaces with respect to nail 60 of FIGS. 1-8; see FIGS. 23-25.

Nail 240 may be structured generally like nail 60. Accordingly, nail 240 may have a leading segment 92 attached to a trailing segment 94 via an adjustable joint 76 having movable and fixed configurations. A locking member 242 may be in threaded engagement with trailing segment 94 and may be turned to advance or retract the locking member, which adjusts the nail between locked and unlocked configurations.

However, nail 240 has noteworthy differences from nail 60. Locking member 242 does not have a spherical head that projects into a spherical cavity of leading segment 92 for capture therein (e.g., compare with FIGS. 7 and 8). Instead, leading segment 92 has a spanning member 244 that spans the adjustable joint. The spanning member has a spherical head 245 that is captured in a cavity 246 defined by trailing segment 94 and formed in part by a flange member 248 that creates a spherical flange at the end of the trailing segment. The flange member is in threaded engagement with a body member 250 of trailing segment 94. Also, locking member 242 bears against the spherical head of spanning member 244 to fix the adjustable joint.

### Example 2

### Articulated Nail with Deformable Locking Member

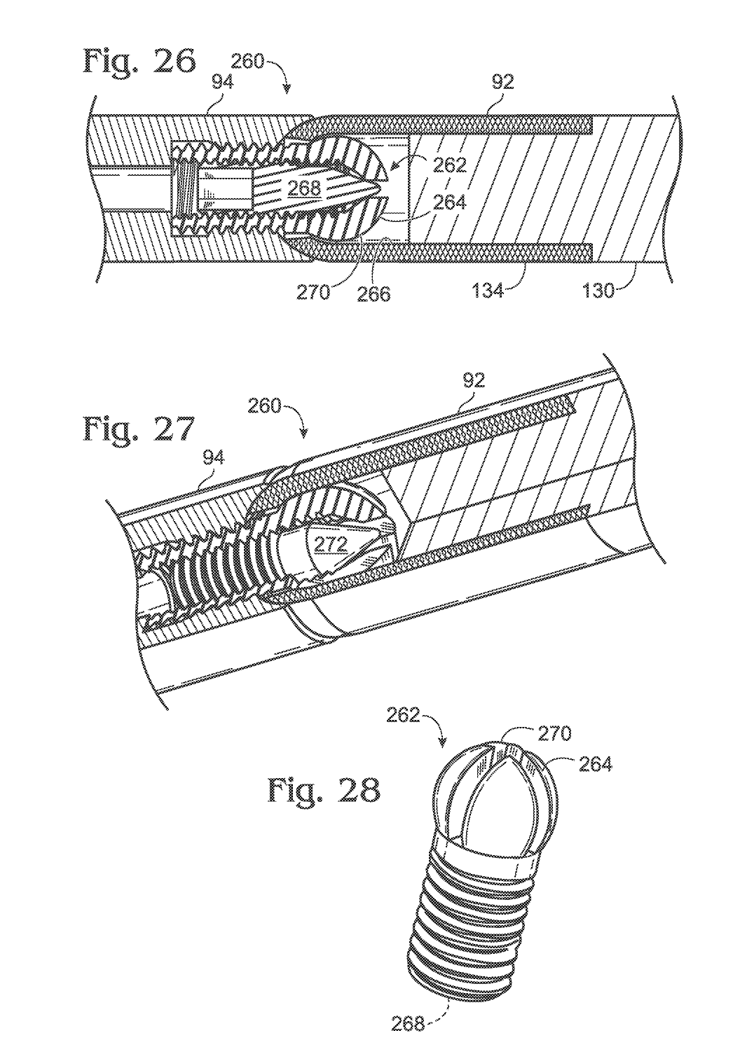

This example describes an exemplary articulated nail 260 having a locking member 262 that is deformable to place the nail in a fixed configuration; see FIGS. 26-28.

Nail 260 has structural similarity to nail 60 of FIGS. 1-8. For example, locking member 262 spans the adjustable joint and has a spherical head 264 that is captured in a cavity 266 defined at the trailing end of leading segment 92.

However, nail 260 has noteworthy differences from nail 60. Locking member 262 is composed of two discrete components, namely, an actuator 268 and an expandable member 270. The actuator is in threaded engagement with the expandable member and has a tapered nose 272 (see FIG. 27). The expandable member includes spherical head 264 (see FIG. 28), which is composed of integral sections that can be urged apart from one another by nose 272, which acts as a wedge when the actuator is advanced. Expansion of spherical head 264 produces frictional engagement of the head with a wall of cavity 266 (see FIG. 26), which locks the nail to prevent pivotal motion at the adjustable joint.

### Example 3

### Articulated Nails with Projections at the Joint Interface of the Nail

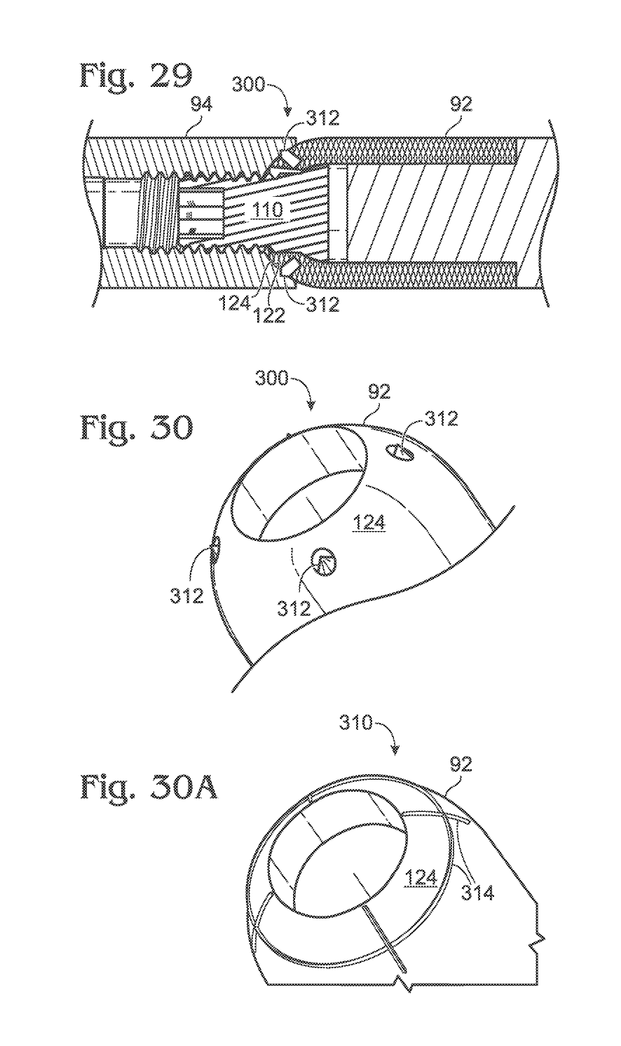

This example describes exemplary articulated nails 300, 310 having projections formed at the joint interface of the nail to resist slippage in the fixed configuration of the joint; see FIGS. 29, 30, and 30A.

FIGS. 29 and 30 show nail 300, which is a modified version of nail 60 (see FIGS. 1-8) having projections 312 formed on outer spherical surface region 124 of the nail. The projections may, for example, be prongs that slightly penetrate adjacent spherical surface region 122 of trailing segment 94 in response to compressive force generated by locking member 110 when the adjustable joint is locked to prevent pivotal motion.

Nail 310 is generally similar to nail 300 except that outer spherical surface region 124 has ribs 314 formed thereon.

### Example 4

### Articulated Nails with a Cam Mechanism

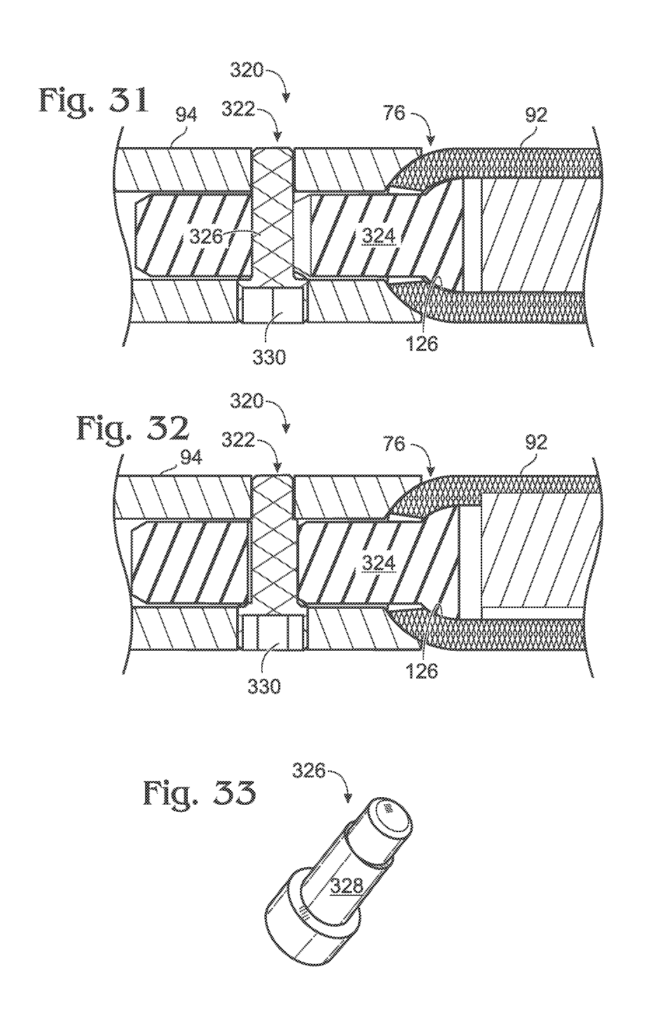

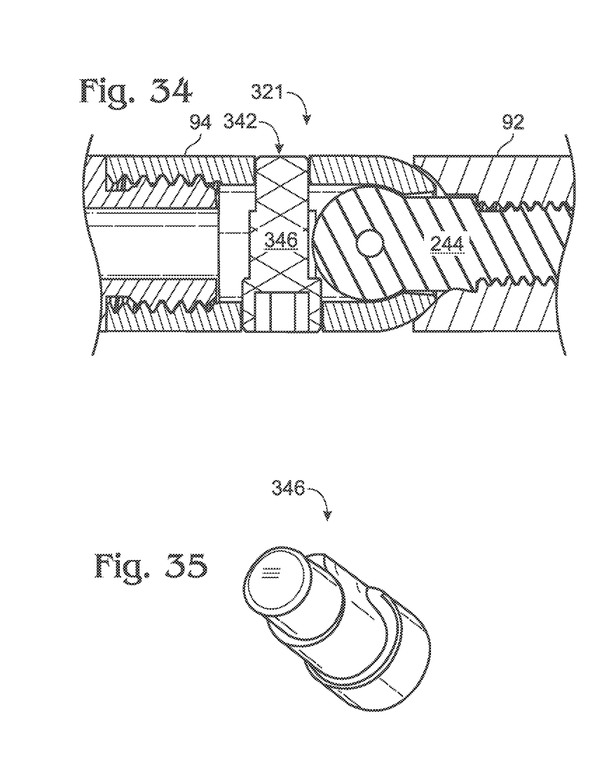

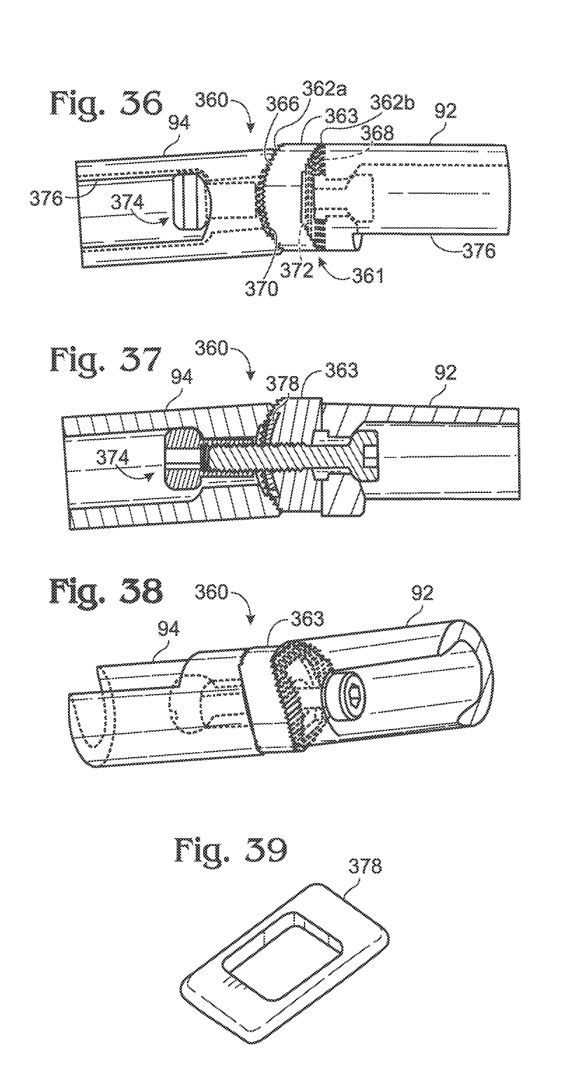

This example describes an exemplary articulated nails 320, 321 having a cam mechanism that is operable to adjust the joint of each nail between a movable (unlocked) and fixed (locked) configuration; see FIGS. 31-35.

FIGS. 31 and 32 show nail 320 in respective unlocked and locked configurations of adjustable joint 76. Nail 320 has a cam mechanism 322 in place of locking member 110 of nail 60 (see FIGS. 2-8). The cam mechanism interchangeably may be termed a locking member or a locking mechanism.

Cam mechanism 322 includes a spanning member 324 that spans the junction between leading segment 92 and trailing segment 94. The spanning member engages wall 126 in the fixed configuration of the adjustable joint, as described above for nail 60.

The longitudinal position of spanning member 324 is controlled by an actuator 326 of the cam mechanism rather than threaded advancement or retraction (as in nail 60). The actuator extends through an opening of the spanning member and is pivotably mounted in the trailing segment of the nail. The actuator has a shaft forming a camming surface 328 disposed asymmetrically about the central long axis of the actuator (see FIG. 33). For example, the shaft may have an elliptical cross section or a circular cross-section that is not concentric with the rotation axis of the actuator shaft. In any event, rotation of the actuator creates longitudinal motion of the spanning member.

The actuator is accessible from a side of the nail. In other words, the actuator has a driver engagement structure 330 disposed adjacent a side surface region of trailing segment 94. Accordingly, the trailing segment of the nail can be attached to bone with fasteners before the nail is bent, without restricting access to driver engagement structure 330.

FIG. 34 shows nail 321, which is more similar to nail 240 of FIGS. 23-25 than nail 60 of FIGS. 1-8. Nail 340 has a cam mechanism 342 that operates generally as described above for cam mechanism 322 of nail 320 (see FIGS. 31-33). However, actuator 346 of cam mechanism 342 bears against spanning member 244, which spans a junction of the segments and is fixed to leading segment 92 (rather than being translatable with respect to both segments as in nail 320).

### Example 5

### Articulated Nails with Discrete Adjustability

This example describes exemplary articulated nails 360, 400, and 480 each having an adjustable joint region with serrated interfaces to provide discrete angular adjustability of nail segments relative to one another with two or three degrees of pivotal freedom, with each degree of freedom provided by a distinct joint; see FIGS. 36-42.

FIGS. 36-38 shows various views of nail 360. The nail includes a leading segment 92 and a trailing segment 94 connected by a joint region 361 having a pair of cylindrical joints 362a, 362b. Each joint may be formed where a spacer 363 contacts one of the nail segments. The spacer may have opposing, curved, generally cylindrical ends each forming a respective set of serrations 366, 368 (interchangeably termed teeth), with each set arranged transverse (e.g., orthogonally) to the other set. The serrations of each set may extend parallel to one another. The serrations mesh, at respective joints 362a and 362b, with complementary sets of teeth 370 and 372 (interchangeably termed serrations) formed by cylindrical ends of the trailing and leading segments, respectively. The teeth within each set 370 or 372 may extend parallel to another and may have the same spacing as serrations 336 and 368, respectively.

A fastener assembly 374 spans the two joints and is adjustable to permit or restrict changes to the meshed engagement of the serrations and the teeth. Adjacent serrations/teeth within a set may represent any suitable angular adjustment of the nail segments relative to each other, such as about 0.25, 0.5, 1, or 2 degrees, among others. In other words, offsetting the meshed engagement at either end of spacer 363 by one tooth may change the angular disposition of one segment relative to the other by no more than about 0.25, 0.5, 1, or 2 degrees, among others.

One of both nail segments may or may not define a lateral window 376 that permits access to the fastener assembly (see FIG. 36). The lateral window may extend any suitable portion of the length of the corresponding nail segment. The trailing segment may or may not define an axial passage that communicates with the fastener assembly.

FIG. 39 shows a biasing member or spring, such as a leaf spring 378, that may be disposed at each segment-spacer interface (i.e., nail 360 may have a pair of the leaf springs, one at each joint). The biasing member can urge the serrations and teeth of the associated joint out of meshed engagement with each other when the nail is disposed in an adjustable configuration, to enable offsetting the meshed engagement during nail adjustment. FIG. 37 shows a leaf spring 378 disposed operatively.

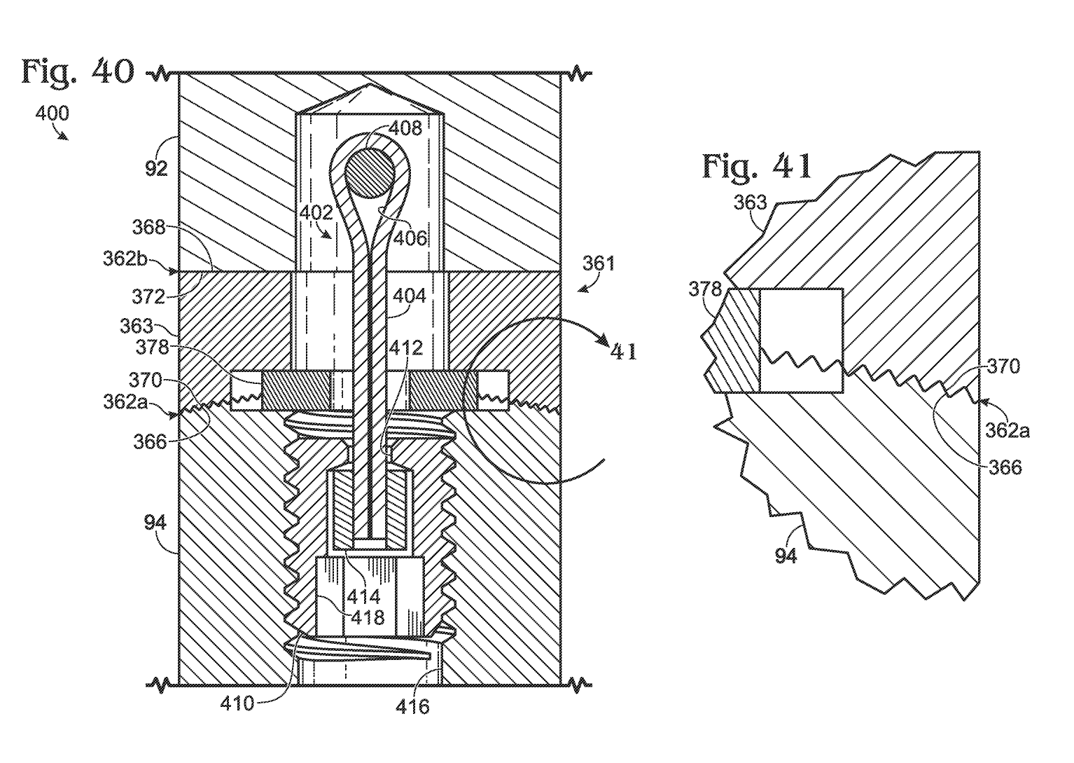

FIG. 40 shows a fragmentary longitudinal sectional view of another exemplary articulated nail 400 with discrete angular adjustment of nail segments 92, 94. The nail may have any of the features of nail 360 or any other nail disclosed herein. For example, the nail may have a single spacer 363 that contacts the rear boundary of leading segment 92 and the front boundary of trailing segment 94. The spacer may form a pair of joints 362a and 362b with the respective nail segments, and each joint may provide mechanical engagement via teeth 366, 368, 370, and 372, as described above for nail 360. Here, however, the teeth are shown as being smaller and more numerous, and arranged along an arc with a larger radius of curvature, which allows the surgeon to make a finer angular adjustment of one nail segment relative to the other nail segment.

Nail segments 92, 94 may be connected to each other with a fastener assembly 402 including a flexible spanning member 404, such as a wire or cable, that spans the joint region of the nail. Spanning member 404 may be doubled back on itself to form a loop 406 at one end for connection to leading segment 92. The loop may, for example, surround a pin 408 attached to the leading segment. The other end of spanning member 404 may extend into a threaded member 410 through an aperture 412 thereof, and may be anchored therein by attachment to a sleeve 414 that cannot pass through aperture 412. Threaded member 410 may be disposed in threaded engagement with an internally threaded, longitudinal bore 416 of trailing segment 94. The threaded member may define a recess 418 to receive a driver for rotation of the threaded member. In particular, the threaded member may be rotationally advanced toward (or away from) leading segment 92 to reduce (or increase) the tension on the spanning member, which unlocks (or locks) joint region 361. In FIG. 40, the threaded member is in a retracted configuration that locks the joint region of the nail.

FIG. 41 is a magnified portion of the sectional view of FIG. 40, taken generally around the region indicated at “41” in FIG. 40.

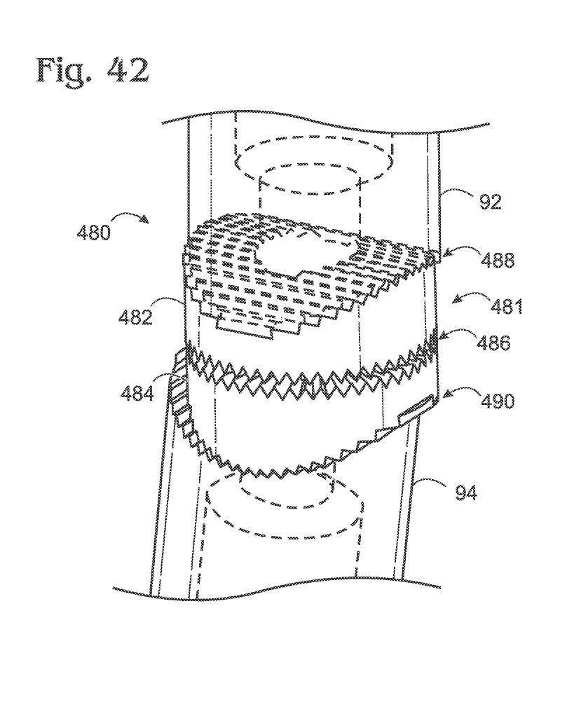

FIG. 42 shows a nail 480 having a joint region 481 formed, in part, by a pair of serrated spacers 482, 484. The spacers may create a central joint 486 with each other, and flanking joints 488, 490 with leading and trailing segments 92, 94 of the nail. The nail segments and spacers 482 and 484 can be held together with, for example, the fastener assembly of nail 360 (see FIG. 36) or nail 400, among others. The utilization of a second spacer allows pivotal adjustment about the long axis of the nail at pivot joint 486, thereby providing three degrees of pivotal freedom (instead of two as in nail 360). More particularly central joint 486 of joint region 481 may have meshed, radially-arranged serrations provided by spacers 482 and 484, to permit pivotal adjustment about the long axis of the nail.

### Example 6

### Exemplary Guide Device

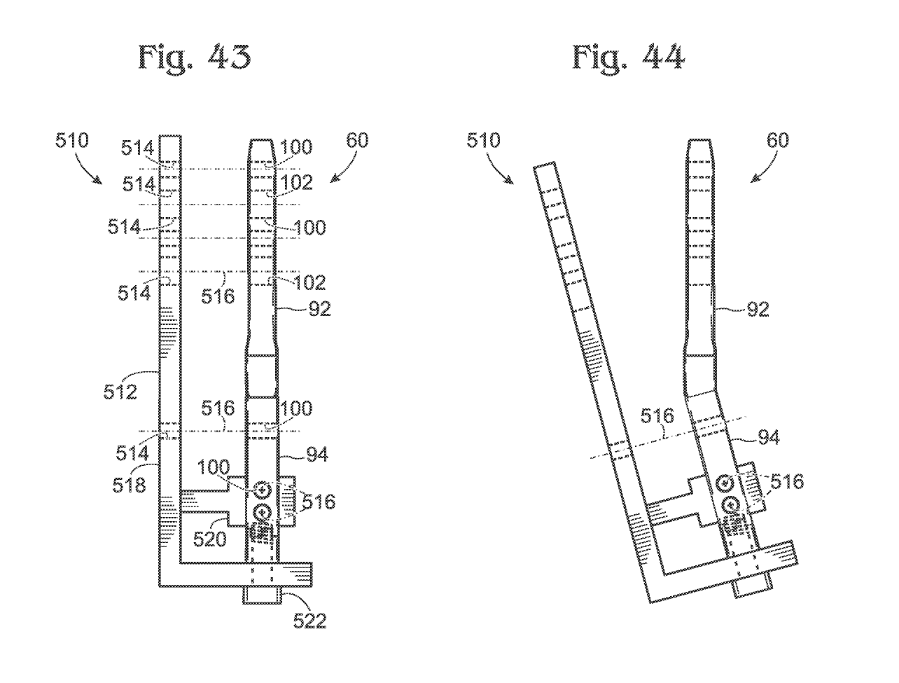

This example describes an exemplary guide device 510 for targeting a hole-forming tool and/or fasteners to apertures of articulated nail 60; see FIGS. 43 and 44. Guide device 510 may be configured to be utilized with any of the nails disclosed herein.

FIG. 43 shows guide device 510 attached to nail 60 with the nail locked in a linear configuration (e.g., as supplied to the surgeon). The guide device may include a body 512 defining openings 514 arranged coaxially, indicated by guide axes 516, with each aperture 100, 102 of the nail. The openings may be used to guide a hole-forming tool (e.g., a drill or punch, among others) to and/or through each of the nail apertures, to create holes in bone to receive fasteners. The openings also or alternatively may be used to guide fasteners into bone and each aperture. The openings of the guide device may be defined by body 512 and/or by, for example, guide tubes (also termed cannulas) extending into and/or through the openings.

Body 512 of the guide device may include a generally L-shaped region 518 and an outrigger region 520 projecting from region 518. The outrigger region may provide openings that align with one or more apertures 100 of trailing region 94.

Guide device 510 may be attached to trailing region 94 of the nail with a guide fastener 522. For example, the guide fastener may extend into the nail from the trailing end thereof for threaded engagement with the nail.

FIG. 44 shows guide device 510 attached to nail 60 with the nail in an angular configuration. Unlike the linear configuration of the nail, the openings of the guide device do not line up with the apertures in leading region 92 of the nail after the nail is bent. In contrast, the apertures in trailing region 94 of the nail are still arranged coaxially with corresponding openings of guide device 510. Accordingly, fasteners may be placed advantageously into the leading segment of the nail, with the aid of the guide device, before the nail is bent or twisted. The guide device may define guides axes that remain aligned with apertures of the nail whether the nail is in a linear or an angular configuration.

### Example 7

### Selected Embodiments

This example presents selected embodiments of the present disclosure related to articulated nails and methods of using articulated nails to fix one or more bones. The selected embodiments are presented as a series of numbered paragraphs.

1. A method of bone fixation, comprising: (A) selecting a nail having first and second segments and an adjustable joint connecting the first segment to the second segment, the adjustable joint having a movable configuration and a fixed configuration; (B) inserting the nail through a calcaneal bone and along a medullary cavity of a tibial bone; (C) bending the inserted nail at the adjustable joint to produce a flexed configuration of the nail; and (D) placing the adjustable joint in the fixed configuration to lock the nail in the flexed configuration.

2. The method of paragraph 1, wherein the nail is inserted through the calcaneal bone and into the tibial bone while the adjustable joint is in a first fixed configuration, and wherein the step of placing disposes the adjustable joint in a second fixed configuration.

3. The method of paragraph 1, wherein the first segment of the nail is inserted into the tibial bone, further comprising a step of attaching the first segment to the tibial bone with one or more fasteners before the step of bending the inserted nail.

4. The method of paragraph 3, further comprising a step of attaching the second segment of the nail to the calcaneal bone with one or more fasteners after the step of placing the adjustable joint in the fixed configuration.

5. The method of paragraph 1, wherein the nail includes a locking member that is manipulable to adjust the nail between the movable configuration and the fixed configuration, and wherein the locking member is accessible from an end of the nail.

6. The method of paragraph 1, wherein the nail includes a locking member that rotates to adjust the nail between the movable configuration and the fixed configuration.

7. The method of paragraph 6, wherein the locking member engages a spherical surface region of one of the segments to place the adjustable joint in the fixed configuration.

8. The method of paragraph 1, wherein the locking member has a spherical surface region that engages one of the segments to place the adjustable joint in the fixed configuration.

9. The method of paragraph 1, wherein the nail is placed in the fixed configuration by operation of a cam mechanism.

10. The method of paragraph 1, wherein the step of placing the adjustable joint in the fixed configuration includes a step of engaging an actuator from a side of the nail.

11. The method of paragraph 1, wherein the nail is placed in the fixed configuration by rotation of a threaded locking member.

12. The method of paragraph 11, wherein the nail includes a threaded locking member that is less than one-half or one-fourth the length of the nail.

13. The method of paragraph 1, wherein the step of inserting the nail disposes the adjustable joint generally between the tibial bone and a talar bone adjacent the tibial bone.

14. The method of paragraph 1, wherein the first segment is an inflexible leading end segment of the nail and the second segment is an inflexible trailing end segment of the nail, and wherein the leading and trailing end segments are attached to each other by the adjustable joint.

15. A method of bone fixation, comprising: (A) selecting a nail having an inflexible leading segment and an inflexible trailing segment collectively constituting at least most of the length of the nail and attached to each other by an adjustable joint having a movable configuration and a fixed configuration; (B) inserting the nail through a calcaneal bone and into a tibial bone; (C) adjusting an orientation of the trailing segment via the adjustable joint such that a relative angular disposition of the calcaneal and tibial bones is changed; and (D) fixing the orientation of the trailing segment by placing the adjustable joint in the fixed configuration.

16. The method of paragraph 15, wherein the step of fixing the orientation includes (1) a step of placing a tool into the nail from a trailing end of the nail and into engagement with a locking member of the nail, and (2) a step of rotating the locking member with the tool.

17. The method of paragraph 16, wherein the locking member includes a spherical surface region, and wherein the spherical surface region engages a spherical surface region of one of the segments to produce the fixed configuration.

18. The method of paragraph 15, wherein the step of fixing the orientation includes a step of operating a cam mechanism of the nail.

19. A fixation device for bone, comprising: an intramedullary nail including an inflexible leading end segment attached by an adjustable joint to an inflexible trailing end segment, the adjustable joint having a movable configuration with at least two degrees of pivotal freedom and a fixed configuration with no pivotal freedom.

20. The fixation device of paragraph 19, further comprising a locking member that engages a spherical surface region of one of the segments to produce the fixed configuration.

21. The fixation device of paragraph 20, wherein the locking member is in threaded engagement with a segment of the nail, and wherein rotation of the locking member moves the locking member longitudinally in the nail.

22. The fixation device of paragraph 21, wherein the locking member is in threaded engagement with the trailing end segment of the nail.

23. The fixation device of paragraph 19, wherein the nail includes a cam mechanism that is operable to adjust the adjustable joint between the movable configuration and the fixed configuration.

24. A method of bone fixation, comprising: (A) selecting the fixation device of any of claims 19-23; (B) inserting the fixation device into a bone having a discontinuity that divides the bone into fragments; (C) bending the fixation device at the adjustable joint to produce a flexed configuration of the nail; and (D) placing the adjustable joint in the fixed configuration to lock the nail in the flexed configuration.

25. The method of paragraph 24, wherein the discontinuity is a fracture of the bone.

26. The method of paragraph 25, wherein the fracture is near an end of a long bone.

27. The method of paragraph 26, wherein the fracture is a fracture of a femoral bone or a tibial bone near a knee joint.

28. A method of bone fixation, comprising: (A) selecting an intramedullary nail having a leading segment and a trailing segment connected by an adjustable joint; (B) inserting at least a portion of the nail longitudinally into a medullary cavity of a bone with the nail locked in a straight configuration; (C) disposing the nail in a bent configuration, after inserting the nail into the bone, by moving at least one of the segments relative to the other segment at the adjustable joint; and (D) locking the nail in the bent configuration.

29. The method of paragraph 28, wherein each of the leading and trailing segments is inflexible.

30. The method of paragraph 28, further comprising a step of attaching the leading segment to the bone with one or more fasteners before the step of disposing the nail in a bent configuration.

31. The method of paragraph 28, wherein the step of inserting disposes the nail in a tibial bone and a calcaneal bone.

32. The method of paragraph 28, further comprising a step of attaching the trailing segment to bone with one or more fasteners after the step of disposing the nail in a bent configuration.

### Example 8

### Further Selected Embodiments

This example presents further selected embodiments of the present disclosure related to articulated nails and methods of using articulated nails to fix one or more bones. The selected embodiments are presented as a series of numbered paragraphs.

1. A fixation system for bone, comprising: a nail having a first segment and a second segment that extend from respective opposite ends of the nail to a joint region that connects the segments, each segment defining one or more transverse apertures configured to receive a fastener that attaches the segment to bone, the joint region having a movable configuration that permits pivotal rearrangement of the segments relative to each other in each of a pair of planes arranged transverse to each other and a locked configuration that fixes the segments relative to each other.

2. The system of paragraph 1, wherein the joint region permits pivotal rearrangement of the segments relative to each other in each of a pair of planes arranged parallel to a long axis of a segment and transverse to each other.

3. The system of paragraph 1 or paragraph 2, wherein each of the first segment and the second segment is nonarticulated, and wherein the segments collectively extend along a majority of the length of the nail.

4. The system of any of paragraphs 1 to 3, wherein each of the first segment and the second segment is only one piece.

5. The system of any of paragraphs 1 to 4, wherein the joint region includes one or more joints, and wherein frictional engagement at each of the one or more joints fixes the nail in the locked configuration.

6. The system of any of paragraphs 1 to 5, wherein the joint region has only one joint that permits the segments to pivot relative to each other.

7. The system of any of paragraphs 1 to 4, 6, and 7, wherein the joint region includes one or more joints, and wherein mechanical engagement at each of the one or more joints fixes the nail in the locked configuration.

8. The system of paragraph 7, wherein the one or more joints include a joint having a pair of mutually engageable surface regions, wherein one of the surface regions defines a plurality of recesses, and wherein the other of the surface regions defines a plurality of projections configured to be received in the plurality of recesses.

9. The system of paragraph 8, wherein one of the surface regions defines one or more teeth that engage one or more teeth of the other surface region when the nail is locked.

10. The system of any of paragraphs 1 to 5 and 7 to 9, wherein the joint region includes a pair of joints arranged along the nail from each other, and wherein each joint permits pivotal adjustment of the segments relative to each other about a single pivot axis.

11. The system of any of paragraphs 1 to 10, wherein a locking member spans the joint region.

12. The system of paragraph 11, wherein the locking member is rigid.

13. The system of paragraph 11, wherein the locking member is flexible.

14. The system of any of paragraphs 1 to 13, further comprising a plurality of threaded fasteners each configured to extend into one of the transverse apertures.

15. The system of any of paragraphs 1 to 14, further comprising a guide device that attaches to one of the segments and defines a guide axis for each of the apertures.

16. The system of any of paragraphs 1 to 15, wherein the nail is placed in the locked configuration by rotation of a threaded member.

17. The system of paragraph 16, wherein the threaded member has a length that is less than one-fourth the length of the nail.

18. A method of bone fixation with a nail having a first segment and a second segment that extend from respective opposite ends of the nail to a joint region that connects the segments, the method comprising: (A) inserting the nail into bone with the nail locked in a linear configuration; (B) attaching each of the segments to bone with one or more fasteners; (C) arranging the nail in an angular configuration, after insertion of the nail into the bone, by moving at least one of the segments relative to the other segment at the joint region; and (D) locking the nail in the angular configuration.

19. The method of paragraph 18, wherein the first segment enters bone before the second segment, and wherein the step of attaching each of the segments to bone includes a step of attaching the first segment to bone before the step of arranging the nail in an angular configuration.

20. The method of paragraph 19, wherein the step of attaching includes a step of attaching the second segment to bone after the step of locking the nail.

21. The method of any of paragraphs 18 to 20, wherein the step of inserting the nail includes a step of positioning at least one of the segments longitudinally in a medullary cavity.

22. The method of any of paragraphs 18 to 21, wherein each of the segments is nonarticulated.

23. The method of paragraph 22, wherein each of the segments is only one piece.

24. The method of any of paragraphs 18 to 23, wherein the joint region permits the segments to be pivoted relative to each other in each of a pair of planes arranged parallel to a long axis of a segment and transverse to each other.

25. The method of any of paragraphs 18 to 24, wherein the joint region includes one or more joints, and wherein frictional engagement at each of the one or more joints locks the nail in the angular configuration.

26. The method of any of paragraphs 18 to 25, wherein the joint region has only one joint that permits the segments to pivot relative to each other.

27. The method of any of paragraphs 18 to 26, wherein the joint region includes one or more joints, and wherein mechanical engagement at each of the one or more joints locks the nail in the linear and angular configurations.

28. The method of paragraph 27, wherein the one or more joints include a joint having a pair of mutually engageable surface regions, wherein one of the surface regions defines a plurality of recesses, and wherein the other of the surface regions defines a plurality of projections configured to be received in the recesses.

29. The method of paragraph 28, wherein one of the surface regions defines one or more teeth that engage one or more teeth of the other surface region when the nail is locked.

30. The method of any of paragraphs 18 to 25 and 27 to 29, wherein the joint region includes a pair of joints arranged along the nail from each other, and wherein each joint permits pivotal adjustment of the segments relative to each other with only one degree of pivotal freedom.

31. The method of any of paragraphs 18 to 30, wherein each segment defines one or more transverse apertures, and wherein the step of attaching each of the segments to bone includes a step of disposing a portion of a fastener in a transverse aperture of each segment.

32. The method of any of paragraphs 18 to 31, wherein a locking member spans the joint region.

33. The method of paragraph 32, wherein the locking member is rigid.

34. The method of paragraph 32, wherein the locking member is flexible.

35. The method of any of paragraphs 18 to 34, wherein the step of inserting the nail disposes a first portion of the nail in a tibial bone and a second portion of the nail in a calcaneal bone.

36. The method of paragraph 35, wherein the step of inserting the nail disposes the pivot region generally between the tibial bone and a talar bone adjacent the tibial bone.

37. The method of any of paragraphs 18 to 34, wherein the step of inserting the nail disposes the nail in only one bone, and wherein the only one bone is fractured.

38. The method of paragraph 37, wherein the bone is a femur, and wherein the step of arranging the nail in an angular configuration is performed with the joint region positioned in a distal region of the femur.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

We claim:

1. A method of bone fixation with a nail having a first segment and a second segment that extend from respective opposite ends of the nail to a joint region that connects the segments, the method comprising:
inserting the nail into bone with the nail locked in a linear configuration;
attaching each of the segments to bone with one or more fasteners;
arranging the nail in an angular configuration, after insertion of the nail into the bone, by moving at least one of the segments relative to the other segment at the joint region; and
locking the nail in the angular configuration.

2. The method of claim 1, wherein the first segment enters bone before the second segment, and wherein the step of attaching each of the segments to bone includes a step of attaching the first segment to bone before the step of arranging the nail in an angular configuration.

3. The method of claim 2, wherein the step of attaching each of the segments to bone includes a step of attaching the second segment to bone before the step of locking the nail.

4. The method of claim 1, wherein the step of inserting the nail includes a step of positioning at least one of the segments longitudinally in a medullary cavity.

5. The method of claim 1, wherein each of the segments is nonarticulated.

6. The method of claim 5, wherein each of the segments is only one piece.

7. The method of claim 1, wherein the joint region permits the segments to be pivoted relative to each other in each of a pair of planes arranged parallel to a long axis of a segment and transverse to each other.

8. The method of claim 1, wherein the joint region includes one or more joints, and wherein frictional engagement at each of the one or more joints locks the nail in the angular configuration.

9. The method of claim 1, wherein the joint region has only one joint that permits the segments to pivot relative to each other.

10. The method of claim 1, wherein the joint region includes one or more joints, and wherein mechanical engagement at each of the one or more joints locks the nail in the angular configuration.

11. The method of claim 10, wherein the one or more joints include a joint having a pair of mutually engageable surface regions, wherein one of the surface regions defines a plurality of recesses, and wherein the other of the surface regions defines a plurality of projections configured to be received in the recesses.

12. The method of claim 11, wherein one of the surface regions defines one or more teeth that engage one or more teeth of the other surface region when the nail is locked.

13. The method of claim 1, wherein the joint region includes a pair of joints arranged along the nail from each other, and wherein each joint permits pivotal adjustment of the segments relative to each other with only one degree of pivotal freedom.

14. The method of claim 1, wherein each segment defines one or more transverse apertures, and wherein the step of attaching each of the segments to bone includes a step of disposing a portion of a fastener in a transverse aperture of each segment.

15. The method of claim 1, wherein a locking member spans the joint region.

16. The method of claim 15, wherein the locking member is rigid.

17. The method of claim 1, wherein the step of inserting the nail disposes a first portion of the nail in a tibial bone and a second portion of the nail in a calcaneal bone.

18. The method of claim 17, wherein the step of inserting the nail disposes the pivot region generally between the tibial bone and a talar bone adjacent the tibial bone.

19. A fixation system for bone, comprising:
a nail having a first segment and a second segment that extend from respective opposite ends of the nail to a joint region that connects the segments, each segment defining one or more transverse apertures configured to receive a fastener that attaches the segment to bone, the joint region having a movable configuration that permits pivotal rearrangement of the segments relative to each other in each of a pair of planes arranged transverse to each other and a locked configuration that fixes the segments relative to each other.
